(19) United States
(12) Patent Application Publication
(10) Pub. No.: US 2025/0072833 A1
(43) Pub. Date: Mar. 6, 2025

(54) TECHNIQUES FOR PROVIDING AUTOMOTIVE-BASED FEEDBACK VIA A WEARABLE DEVICE

(71) Applicant: Oura Health Oy, Oulu (FI)

(72) Inventors: Nicolas Jean Bernard Beuchat, Lausanne (CH); Harry Storbacka, Oulu (FI); Ketal Narendra Patel, Cupertino, CA (US); Mika Erkkila, Oulu (FI); Krystle Elaine de Mesa, Redwood City, CA (US); Kami Barnes, Weymouth, MA (US); Nicholas Moretto, Reno, NV (US); Chetan Bangalore Chikkamariyappa, Dublin, CA (US); Ryan Hall, Oceanside, CA (US)

(21) Appl. No.: 18/824,725

(22) Filed: Sep. 4, 2024

Related U.S. Application Data
(60) Provisional application No. 63/580,651, filed on Sep. 5, 2023.

(51) Int. Cl. A61B 5/00; A61B 5/01; A61B 5/16; B60Q 9/00; G01C 21/34; H04B 1/3827; H04W 4/40; H04W 4/90

(52) U.S. Cl. CPC A61B 5/6826; A61B 5/0002; A61B 5/01; A61B 5/165; A61B 5/486; A61B 5/746; B60Q 9/00; G01C 21/3484; H04B 1/385; A61B 2503/22; A61B 2562/0271; H04B 2001/3861; H04W 4/40; H04W 4/90

(57) ABSTRACT

Methods, systems, and devices for leveraging automotive and wearable-based data are described. For example, a system may include a wearable device, a user device associated with the wearable device, and a vehicle. The system may determine when a user is within a proximity of the vehicle based on communications between the vehicle and the wearable device, between the vehicle and the user device, or both. The system may retrieve physiological data measurement from the user via the wearable device based on determining the user is positioned in a proximity of the vehicle, such that one or more operational parameters of the vehicle may be adjusted based on the physiological data. Additionally, or alternatively, the system may receive telemetry data from one more sensors of the vehicle and may provide feedback to the user based on the telemetry data satisfying one or more trigger conditions.

### CROSS REFERENCE

The present Application for Patent claims the benefit of U.S. Provisional Patent Application No. 63/580,651 by Beuchat et al., entitled “TECHNIQUES FOR LEVERAGING AUTOMOTIVE AND WEARABLE-BASED DATA,” filed Sep. 5, 2023, assigned to the assignee hereof, and expressly incorporated by reference herein.

### FIELD OF TECHNOLOGY

The following relates to wearable devices and data processing, including techniques for leveraging automotive and wearable-based data.

### BACKGROUND

Some wearable devices may be configured to collect data from users associated with a physiological state of the user, a psychological state of the user, actions performed by the user, or the like thereof. Additionally, the wearable device, or a user device paired with the wearable device, may be configured to provide feedback to the user based on the collected data. However, the data collected from the user and the feedback provided to the user may be limited to a system including the user, the wearable device, and the user device paired with the wearable device.

### BRIEF DESCRIPTION OF THE DRAWINGS

FIG. 1 illustrates an example of a system that supports techniques for leveraging automotive and wearable-based data in accordance with aspects of the present disclosure.

FIG. 2 illustrates an example of a system that supports techniques for leveraging automotive and wearable-based data in accordance with aspects of the present disclosure.

FIG. 3 shows an example of a system that supports techniques for leveraging automotive and wearable-based data in accordance with aspects of the present disclosure.

FIGS. 4 through 6 show flowcharts illustrating methods that support techniques for leveraging automotive and wearable-based data in accordance with aspects of the present disclosure.

### DETAILED DESCRIPTION

A user may use a device (e.g., a wearable device) to determine physiological measurements of the user, such as temperature, heart rate, respiratory rate, photoplethysmography (PPG) data, and the like. Physiological data collected via a wearable device may be used to gain insights into the user's sleeping patterns and overall health. For example, motion data and temperature data may be used to evaluate a user's sleep to determine how restful the sleep is, and how beneficial (or detrimental) the user's sleep is to their overall health. Additionally, the wearable device, or a user device paired with the wearable device, may provide feedback to the user based on the physiological data. However, the physiological data collected from the user and the feedback provided to the user may be limited to a system including the user, the wearable device, and the user device paired with the wearable device. As such, capabilities of the wearable device may be limited to the system.

Accordingly, aspects of the present disclosure are directed to systems and methods which enable an automobile, or vehicle, associated with the user to be incorporated into the system including the wearable ring device to enable physiological data collected via wearable devices to be utilized to selectively control the vehicle and to enable the vehicle to provide feedback to the user. For example, the system may determine when the user is within a proximity of the vehicle based on communications between the vehicle and the wearable device, between the vehicle and the user device, or both. As such, the system may retrieve physiological data measurement from the user via the wearable device based on determining the user is positioned in a proximity of the vehicle, such that one or more operational parameters of the vehicle may be adjusted based on the physiological data. For instance, the system may recognize the user within the vehicle, and may adjust the seat settings, mirror settings, and interior climate control settings based on the user's personalized preferences.

Additionally, or alternatively, the system may receive telemetry data from one more sensors of the vehicle based on the user driving the vehicle and may provide feedback to the user based on satisfaction of a trigger condition, where the trigger condition is based on the telemetry data. For example, the vehicle may recognize that there is another car in the user's blind spot as the user is attempting to change lanes, and may cause the wearable device of the user to provide feedback (e.g., haptic feedback, audio feedback, visual feedback) to alert the user of the additional vehicle in the blind spot. By way of another example, the system may determine that the user is approaching an upcoming turn to reach their destination, and may cause the wearable device to provide feedback to the user to remind the user of their upcoming turn.

Aspects of the disclosure are initially described in the context of systems supporting physiological data collection from users via wearable devices. Aspects of the disclosure are further illustrated by and described with reference to apparatus diagrams, system diagrams, and flowcharts that relate to techniques for leveraging automotive and wearable-based data.

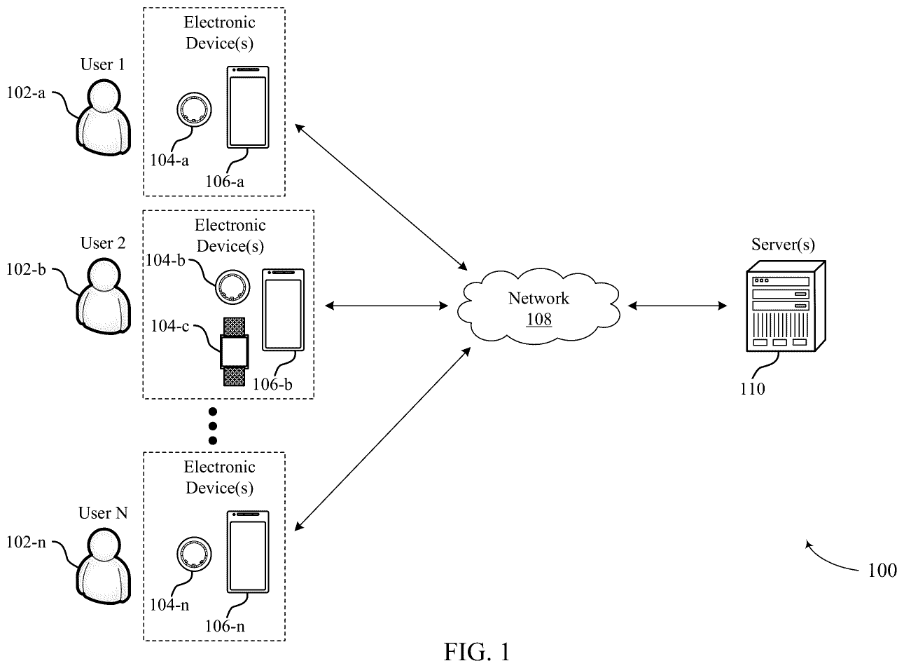

FIG. 1 illustrates an example of a system 100 that supports techniques for leveraging automotive and wearable-based data in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the car, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for leveraging automotive and wearable-based data. For example, the system 100 may additionally include vehicles associated with each user 102. As such, the system 100 may support utilization of physiological data collected via the wearable devices 104 to selectively control the vehicle and utilization of data collected via the vehicle to provide feedback to the respective user 102.

For example, the system 100 may determine when a user 102, such as the user 102-a, is within a proximity of a respective vehicle based on communications between the vehicle and the wearable device 104, such as the ring 104-a, via the network 108, between the vehicle and the user device 106, such as the user device 106-a, via the network 108, or both. As such, the system 100 may retrieve physiological data measurement from the user 102-a via the ring 104-a based on determining the user 102-a is positioned in a proximity of the vehicle, such that one or more operational parameters of the vehicle may be adjusted based on the physiological data. Additionally, or alternatively, the system 100 may receive telemetry data from one or more sensors of the vehicle based on the user 102-a driving the vehicle and may provide feedback to the user 102-a based on satisfaction of a trigger condition, where the trigger condition is based on the telemetry data.

In additional or alternative implementations, vehicles, wearable devices 104, and/or user devices 106 associated with the different respective users 102 may communicate with one another, where such communications may be leveraged to further identify relationships, trends, and physiological conditions within the system 100. For example, vehicles associated with different users may be configured to communicate with one another, and the communications between the vehicles may be leveraged to provide feedback to the respective users 102 (e.g., vehicles may communicate with one another to indicate traffic jams, sudden stops, etc., and the system 100 may use such communications to provide feedback or messages to the users).

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

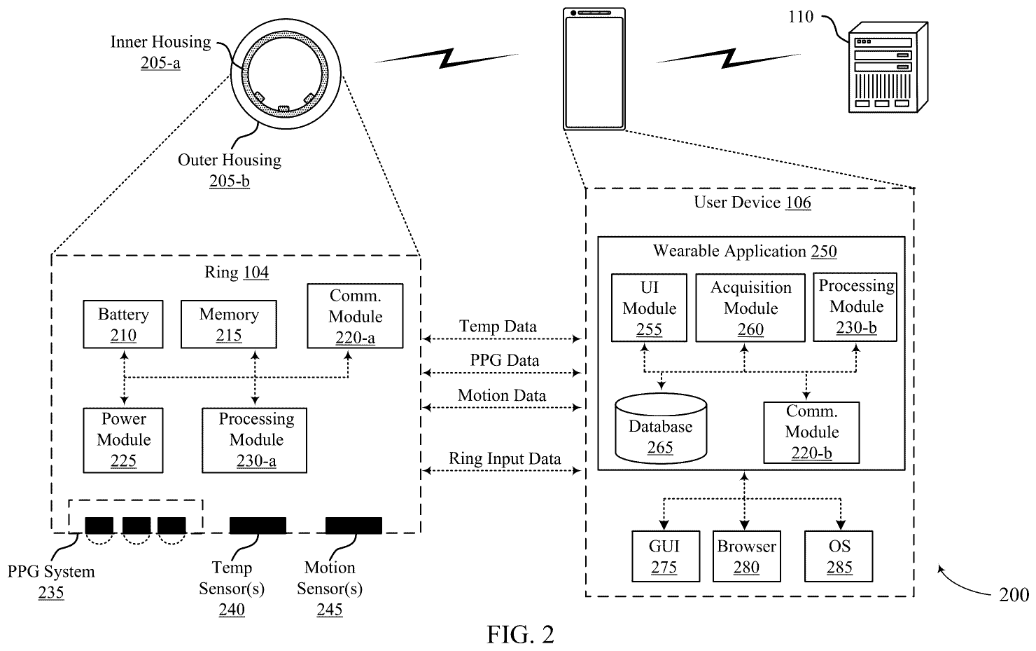

FIG. 2 illustrates an example of a system 200 that supports techniques for leveraging automotive and wearable-based data in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the respective devices of the system 200 may support techniques for leveraging automotive and wearable-based data. For example, the system 100 may additionally include a vehicle associated with a user 102 associated with the ring 104. As such, the system 200 may support utilization of physiological data collected via the ring 104, such as via the PPG system 235, the temperature sensors 204, the motion sensors 245, or any combination thereof, to selectively control the vehicle and utilization of data collected via the vehicle to provide feedback to the respective user 102.

For example, the system 200 may determine when a user 102, such as the user 102-a, is within a proximity of a respective vehicle based on communications between the vehicle and the ring 104, via the communication module 220-a and a communication module 220-c (e.g., not depicted) associated with the vehicle, between the vehicle and the user device 106, via the communication module 220-b and the communication module 220-c, or both. As such, the system 200 may retrieve physiological data measurement from the user 102 via the ring 104 based on determining the user 102 is positioned in a proximity of the vehicle, such that one or more operational parameters of the vehicle may be adjusted based on the physiological data.

Additionally, or alternatively, the system 200 may receive telemetry data from one or more sensors of the vehicle based on the user 102 driving the vehicle and may provide feedback to the user 102 based on satisfaction of a trigger condition, where the trigger condition is based on the telemetry data.

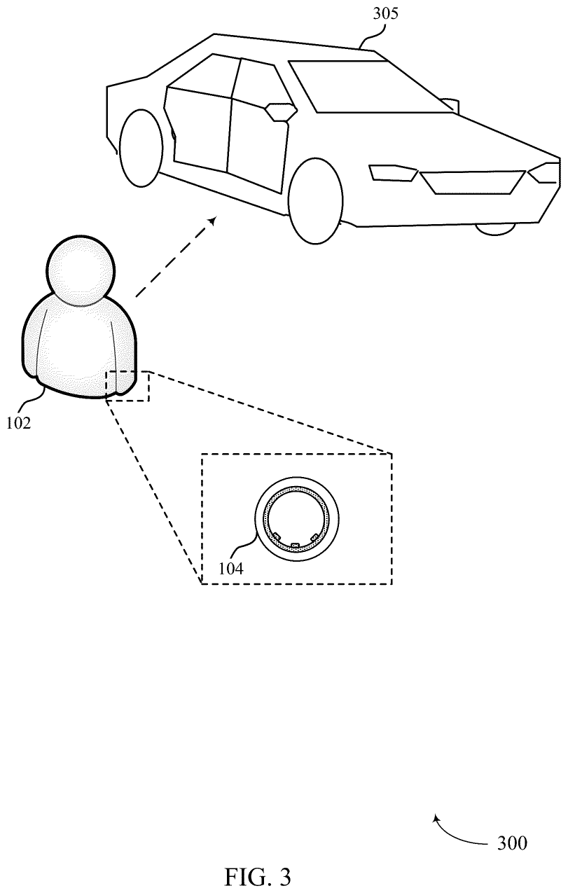

Attendant advantages achieved by leveraging both physiological data collected via the wearable device 104 and additional data collected via a vehicle may be further shown and described with reference to FIG. 3.

FIG. 3 shows an example of a system 300 that supports techniques for leveraging automotive and wearable-based data in accordance with aspects of the present disclosure. Aspects of the system 300 may implement, or be implemented by, aspects of the system 100, the system 200, or both.

In some cases, the system 300, including a user 102, a wearable device 104 (e.g., wearable ring device 104, a finger-worn wearable ring device) associated with the user 102, a user device 106 associated with the wearable device 104, and any other ancillary components supporting the wearable device 104 and the user device 106, may be extended to include a vehicle 305. In other words, the vehicle 305 may be embedded in the system 300, such that data associated with or collected by the wearable device 104, data associated or collected by the vehicle 305, or both, may be utilized to expand the capabilities of the system 300.

In some cases, the wearable device 104, or data collected by the wearable device 104, may be utilized to control the vehicle 305. For example, the system 300 may first determine that the user 102 is positioned, or located, near (e.g., within a threshold proximity of, within a determined proximity of) the vehicle 305 to enable the wearable device 104 to control the vehicle 305. In some cases, the system 300 may determine the user 102 is located near the vehicle 305 based on communications between the wearable device 104 and the vehicle 305, between communications between the user device 106 and the vehicle 305, or both. For example, the wearable device 104, the user device 106, the vehicle 305, or any combination thereof, may include one or more sensors, such as a proximity sensor or a GPS system, that enables the system 300 to determine when the user 102 is located near the vehicle 305. Additionally, or alternatively, signaling may be transmitted between the wearable device 104 (and/or user device 106) and the vehicle 305, such that the signaling, or timing associated with the signaling, may be used by the system 300 to determine when the user 102 is located near the vehicle 305.

As such, the system 300 may retrieve physiological data measured from the user 102 via the wearable device 104 based on determining the user 102 is located near the vehicle 305 and may transmit, to the vehicle 305 (e.g., from the wearable device 104, from the user device 106, or both), a signal to control the vehicle 305 in one or more ways (e.g., via Bluetooth, Near Field Communications (NFC), or both). That is, the signal may cause the vehicle to adjust one or more operational parameters of the vehicle 305 based on the physiological data.

For example, the vehicle 305 may unlock based on determining the user 102 is near (or approaching) the vehicle 305 and confirming an identity of the user 102 based on data collected via the wearable device 104. That is, the system 300 may build a user profile associated with the user 102 based on physiological data collected by the wearable device 104 worn by the user 102. As such, as the user 102 (e.g., wearing the wearable device 104) approaches the vehicle 305 (e.g., is within proximity of the vehicle 305), the system 300 may collect current (e.g., or recent, within a threshold duration prior to the user 102 being within the proximity of the vehicle 305) physiological data associated with the user 102 via the wearable device 104 and compare the current physiological data to the user profile associated with the user 102 (e.g., where the user profile indicates a baseline physiological data associated with the user 102 collected over a duration). If the current physiological data (e.g., one or more of heart rate, HRV, temperature, blood pressure, etc.) matches the user profile associated with the user 102 (e.g., within a threshold tolerance), the system 300 may authenticate, or confirm, the identity of the user 102, and may thereby determine that the user 102 is allowed to access the vehicle 305 (e.g., allowed access to one or more features of the vehicle 305). As such, the system 300 may unlock the vehicle 305 based on the authentication. Conversely, if the current physiological data does not match the user profile associated with the user 102 (e.g., within a threshold tolerance), the system 300 may fail to authenticate, or confirm, the identity of the user 102, and may refrain from unlocking the vehicle 305. In such cases, the system 300 may transmit an alert to one or more external systems associated with the user profile, where the alert indicates that an unauthenticated user 102 attempted to access the vehicle 305. Additionally, or alternatively, the system 300 may authenticate, or confirm, the identity of the user 102 via matching the current physiological data of the user 102 with the user profile associated with the user 102 prior to enabling one or more capabilities of the vehicle 305, such as turning on the vehicle 305. For example, a user 102 may not be able to drive a vehicle for a rideshare service until the identity of the user 102 is authenticated.

In some examples, after matching the current physiological data of the user 102 with the user profile associated with the user 102 (e.g., within the threshold tolerance), the system 300 may request a second form of authentication prior to authenticating the user 102 (e.g., prior to allowing the user 102 to access the vehicle 305). For example, the system 300 may prompt the user to input a code (e.g., a PIN, a set of taps) via a display on the vehicle 305, via the wearable device 104, via the user device 106, or any combination thereof, may prompt the user 102 to perform a gesture, or the like thereof. In such cases, the system 300 may authenticate the user 102 (e.g., allow access to the one or more features of the vehicle) based on matching the current physiological data of the user 102 with the user profile associated with the user 102 and based on the second form of authentication (e.g., provided by the user 102) being valid. Conversely, the system 300 may fail to authenticate the user 102 (e.g., deny access to the one or more features of the vehicle) based on the second form of authentication (e.g., provided by the user 102) being invalid, regardless of whether (e.g., even if) the system 300 matched the current physiological data of the user 102 with the user profile associated with the user 102.

In some examples, the user 102 may grant access to the vehicle 305 to an additional user 102. That is, the system 300 may generate an additional user profile associated with the additional user 102. For example, the additional user 102 may be a rideshare passenger, such that the vehicle 305 may not allow the additional user 102 to gain entry in the vehicle 305 until an identity of the additional user 102 is authenticated (e.g., matches a user profile associated with the user 102 in a rideshare application). As such, the system 300 may use the techniques described herein to authenticate the additional user 102 and allow or deny access to the vehicle 305 based on the authentication and authorization of the user 102 that owns the vehicle 305. Additionally, or alternatively, in some cases, the system 300 may activate or deactivate vehicle access based on physiological data (e.g., for the user 102, for any additional users 102, or both). For example, if the vehicle 305 is stolen, the user 102 may lock the vehicle 305 remotely and may deactivate vehicle access based on physiological data. Additionally, or alternatively, the system 300 may enable the user 102 to activate or deactivate physiological data-based authentication of users 102 related to access of one or more features of the vehicle.

In some aspects, the vehicle 305 may select a navigational route based on physiological data associated with the user 102. For example, the system 300 may receive an indication of a destination of the user 102 and an indication of a desire of the user 102 to receive directions to navigate to the destination. As such, the system 300 may retrieve physiological data associated with the user 102 to determine a physiological state, psychological state, or both, of the user 102 to aid in route selection. That is, the system 300 may identify a fastest route between the user 102 and the desired destination, and may modify the fastest route based on the physiological state, the psychological state, or both, of the user 102 (e.g., state of consciousness). For example, the system 300 may determine, based on the collected physiological data, that the user 102 is stressed. As such, the system 300 may select a route that may be longer than the fastest route, but has less traffic or includes one or more features to aid in relaxation of the user 102 (e.g., a more scenic view). In another example, the system 300 may determine that the user 102 is feeling nauseous and may select a route that contains less curves, a smoother road surface, avoids areas with distinct smells (e.g., avoids driving by a landfill), or the like thereof. In another example, the system 300 may determine, during route selection or while the user is traveling a selected route, that the user 102 is fatigued or is drowsy (e.g., tired) and may select a route or modify a route, respectively, to include one or more rest stops or breaks.

In some cases, the vehicle 305 may mute one or more notifications via the vehicle 305 based on physiological data associated with the user 102. For example, as described previously, the system 300 may retrieve physiological data associated with the user 102 to determine a physiological state, psychological state, or both, of the user 102 (e.g., state of consciousness). As such, the vehicle 305 may determine whether to allow a notification or mute a notification via the vehicle 305 based on the physiological state, the psychological state, or both, of the user 102. For example, the system 300 may determine, based on the collected physiological data, that the user 102 is stressed or is not concentrating (e.g., is experiencing a decreased level of concentration). As such, the vehicle 305 may mute (e.g., silence) notifications (e.g., indications) of incoming calls, incoming messages, or both, while driving to enable the user 102 to avoid distractions and reduce stress levels. In some cases, a subset of the notifications of incoming calls, incoming messages, or both, may not be muted based on a type of the subset of notifications, an additional user 102 associated with the subset of notifications, or both. For example, notifications associated with a user-selected set of contacts (e.g., additional users 102), notifications associated with one or more types of notifications (e.g., emergency notifications), or both, may be allowed by the system 300, while other notifications not associated with the user-selected set of contacts, the one or more types of notifications, or both, may be muted by the system 300. In some cases, the subset of notifications may not be muted based on the collected physiological data indicating that a stress level of the user 102, a concentration level of the user 102, or both, satisfying a respective threshold (e.g., being less than a threshold stress level, being greater than a threshold concentration level), indicating that the user 102 is capable of handing the distraction with minimal impacts to their driving ability.

In some cases, the concentration level of the user 102 may be based on HRV data associated with the user 102 (e.g., the physiological data includes the HRV data. For example, a first range of HRV values (e.g., less than a first threshold HRV) may be associated with a first concentration level (e.g., not concentrated on driving, concentrated on driving), a second range of values (e.g., between the first threshold HRV and a second threshold HRV) may be associated with a second concentration level (e.g., somewhat concentrated on driving), and a third HRV within a third range of values (e.g., greater than the second threshold HRV) may be associated with a third concentration level (e.g., concentrated on driving, not concentrated on driving). Additionally, or alternatively, the concentration level of the user 102 may be based on current physiological data associated with the user 102 relative to baseline physiological data associated with the user 102. For example, the system 300 may determine that the user 102 is not concentration (e.g., is associated with a concentration level below the threshold concentration level) based on a difference between the current physiological data and the baseline physiological data (e.g., exceeding a threshold difference).

Additionally, or alternatively, the vehicle 305 may disable or modify one or more capabilities, or functions, of the vehicle 305 based on physiological data associated with the user 102. For example, the system 300 may determine that the user 102 is experiencing an altered state of consciousness (e.g., is intoxicated, sleepy, etc.) based on physiological data associated with the user 102, such that the vehicle 305 may prevent the user 102 from operating the vehicle 305 (e.g., will not unlock the vehicle 305, will not start the vehicle 305, etc.) or alert the user 102 that it is not recommended for them to drive. For example, the system 300 may determine that the user 102 has been drinking (e.g., based on tags for alcohol, gesture recognition of drinking, a location of the user 102 being at a bar, elevated heart rate, an abnormal gait of the user 102, detecting ethanol in sweat of the user 102) and may alert the user 102 that the user 102 is possibly unfit to drive and may suggest that the user 102 contact a rideshare service (e.g., Uber, Lyft, taxi service, or the thereof). In some cases, the system 300 may further disable an ability for the user 102 to start the vehicle 305 (e.g., prevent the vehicle 305 from starting), may alert or contact (e.g., via the user device 106, the vehicle 305, or both) a designated driver associated with the user, or both. In another example, the system 300 may determine that the user 102 is tired, or sleepy, such that the vehicle 305 may disable an ability for the user 102 to receive, accept, or reply to calls or messages via the vehicle 305, such that the user 102 may increase focus and avoid distractions while driving. Additionally, or alternatively, the system 300 may detect if the user 102 begins to fall asleep while driving and may emit an alarm via the vehicle 305, the user device 106, or the wearable device 104 to wake the user.

In some cases, the vehicle 305 may engage automatic driving features in cases where physiological data collected via the wearable device 104 indicates that the user is experiencing an altered state of consciousness (e.g., intoxication, anxiety attack), is becoming drowsy, is experiencing a medical event, etc. For example, the system 300 may automatically park the vehicle 305, stop the vehicle 305 (e.g., in a safe location), or drive the vehicle to a hospital (e.g., for self-driving vehicles 305) based on detecting that the user 102 is experiencing a medical event (e.g., severe medical event). Additionally, or alternatively, the system 300 may alert (e.g., via the user device 106, the vehicle 305, or both) emergency medical services, one or more emergency contacts, or both, and may provide a location of the user 102 (e.g., and the vehicle 305), an indication of physiological data associated with the user 102 (e.g., a type of medical event, information associated with vitals of the user, etc.), or both, through the alert. Additionally, or alternatively, the system 300 may alert the user 102 (e.g., via the vehicle 305) to pull over based on detection of the medical event (e.g., if the medical event is less severe).

Additionally, or alternatively, the vehicle 305 may modify one or more operational parameters, or user settings, of the vehicle 305 based on physiological data associated with the user 102. The one or more operational parameters may include a vehicle seat setting (e.g., seat position, lumbar support, recline angle), a mirror setting, a vehicle climate control setting (e.g., temperature, humidity), an interior lighting setting, a driving performance profile associated with the vehicle 305 (e.g., driving settings of a self-driving vehicle 305), a media setting (e.g., radio stations, volume settings, bass settings, etc.), or any combination thereof. For example, the user profile associated with the user 102, as described previously herein, may include an indication of one or more desired operational parameters for the user 102. As such, the system 300 may determine that the user 102 is approaching the vehicle 305 (e.g., as described previously) and may identify that the user 102 is associated with a user profile. As such, the vehicle 305 may modify one or more operational parameters of the vehicle 305 to match the one or more desired operational parameters associated with the user profile (e.g., prior to or after the user 102 enters the vehicle 305).

In some cases, the one or more desired operational parameters for the user 102 may be dependent on a type (e.g., make or model) of the vehicle 305, a position of the user 102 within the vehicle 305, or both. For example, the user profile associated with the user 102 may indicate that a first set of desired operational parameters is associated with a first type of vehicle 305 (e.g., a truck) and a second set of desired operational parameters is associated with a second type of vehicle 305 (e.g., a sedan). Additionally, or alternatively, the user profile associated with the user 102 may indicate that the first set of desired operational parameters is associated with the user 102 being a driver of the first type of vehicle 305, a third set of desired operational parameters may be associated with the user 102 being a front seat passenger (e.g., co-pilot) of the first type of vehicle 305, and a fourth set of desired operational parameters may be associated with the user 102 being a backseat passenger of the vehicle 305.

Additionally, or alternatively, the one or more desired operational parameters for the user 102 may be dependent on a physiological state of the user 102, a psychological state of the user 102, or both. For example, the user profile may indicate that a fifth set of desired operational parameters may be associated with the user 102 being stressed (e.g., associated with a stress mode of the vehicle 305), a sixth set of desired operational parameters may be associated with the user 102 being drowsy (e.g., associated with a drowsy mode of the vehicle 305), and a seventh set of desired operational parameters may be associated with the user 102 being relaxed (e.g., associated with a relaxed mode of the vehicle 305). Thus, the system 300 may determine whether the user is stressed, drowsy, or relaxed on the physiological data associated with the user 102 and may adjust the one or more desired operational parameters for the user 102 in accordance with a mode associated with the physiological state of the user 102 (e.g., activate the stress mode, the drowsy mode, or the relaxed mode).

In some examples, the system 300 may identify user profiles associated with a driver of the vehicle 305 and associated with one or more passengers of the vehicle 305. That is, the system 300 may identify that there are multiple individuals (or users 102) within the vehicle 305. As such, the system 300 may identify a location of each of the multiple individuals within the vehicle 305 and modify one or more operational parameters within a threshold proximity of each location based on the respective user profile of the respective individuals. For example, the vehicle 305 may modify a temperature of air being blown at the driver's position according to a user profile associated with a first user 102 identified as the driver (e.g., based on a location of the first user 102 being in the driver's position) and may modify a temperature of air being blown at the co-pilot position (e.g., front seat passenger position) according to a user profile associated with a second user 102 identified as a passenger (e.g., based on a location of the second user 102 being in the co-pilot position). In other words, the system 300 (e.g., one or more servers associated with the system 300, the vehicle 305, a user device 106 associated with the system 300, one or more external devices associated with the system 300, or any combination thereof) may store multiple user profiles associated with the vehicle 305 and may match physiological data associated with each user 102 (e.g., the driver and the one or more passengers) to a user profile form the multiple user profiles. In some cases, the system 300 may generate a default user profile associated with a default set of operational parameters for users 102 that are not associated with a wearable device 104 (e.g., users 102 not associated with physiological data).

In some cases, the system 300 may determine when to modify one or more operational parameters, or user settings, of the vehicle 305 in accordance with the user profile associated with the user 102 based on a routine of the user 102. For example, the system 300 may determine that the user 102 has woken up and may cause the vehicle 305 to turn on and to begin heating up to a desired temperature (e.g., associated with the user 102 based on the user profile), to active seat warmers, or both, a threshold duration after the user 102 woke up. In such cases, the threshold duration may be based on a typical morning routine of the user 102. For example, the system 300 may identify that the user 102 typically operates their vehicle 305 an hour after waking, 15 minutes after consuming breakfast, or the like thereof.

Additionally, or alternatively, the vehicle 305 may modify one or more operational parameters, or user settings, of the vehicle 305 based on current physiological data associated with the user 102. For example, the system 300 may identify a temperature of the user 102, a heart rate of the user 102, or both, and may adjust a temperature of the vehicle 305 based on the temperature of the user 102, the heart rate of the user 102, or both. In some examples, the system 300 may modify the temperature of the vehicle 305 based on historical data associated with the user 102. For example, the system 300 may identify that, during a previous driving occasion, raising a temperature of the vehicle 305 by a first value resulted in an increase in a temperature of the user 102 by a second value. In another example, the system 200 may identify that, while located at home, decreasing a temperature of the home by a third value resulted in a decrease in a temperature of the user by a fourth value. As such, the system 300 may identify one or more relationships between changes in temperature (or other parameters/characteristics) of the surroundings of the user 102 and changes in temperature of the user 102 based on the historical data, such that the vehicle 305 may use the one or more relationships to determine how to modify a temperature of the vehicle 305 based on current physiological data associated with the user 102. In another example, the system 300 may identify that the user 102 is in a slouched position (e.g., due to stress, lack of concentration, or muscle tension) and may increase lumbar support of the seat of the user 102, activate a massage function of the seat, or or both.

In another example, the system 300 may adjust one or more operational parameters associated with the vehicle 305 based on a physiological state of the user 102, a psychological state of the user 102, or both. For example, the system 300 may determine that the user 102 is sleepy, or feeling tired, and may increase a volume of music, decrease a temperature of the vehicle 305, cause the seat of the user 102 to vibrate, provide a visual alert to the user 102, or any combination thereof, to help the user 102 stay alert. In some cases, the system 300 may determine that the user 102 is sleepy, or tired, based on a Sleep Score of the user 102 from a previous night, based on electrodermal data collected via the ring 104 (e.g., via one or more electrodermal activity sensors). In another example, the system 300 may determine that the user 102 is stressed and may increase a temperature of the vehicle 305, emit a scent (e.g., aromatherapy) via the cooling/heating system of the vehicle 305, decrease a brightness of lights and displays, decrease a volume of music, prompt the user 102 to perform a relaxation exercise (e.g., listen to a calming podcast, perform a relaxation session, or the like thereof), or any combination thereof, to help the user 102 relax. In some examples, the system 300 may determine that the user 102 is stressed (e.g., or angry or frustrated) based on the user 102 being associated with a high heart rate and a low HRV. As an illustrative example, the vehicle 305 may be a self-driving vehicle 305, such that the relaxation session may instruct the user 102 to recline their seat, take deep breathes, and focus on a relaxing soundscape produced through media of the vehicle 305. In another example, the system 300 may determine that the user 102 is exercising (e.g., at a location that they drove to) and may pre-emptively reduce a temperature of the vehicle 305 to prepare the vehicle 305 to be driven by the user 102 post-exercise. In some cases, the system 300 may determine that the user 102 is exercising based on activity data associated with the user 102, one or more tags input by the user 102, a heart rate of the user 102, sweat detection on the user 102 (e.g., via one or more electrochemical sensors, one or more sweat patches, or both). In some cases, the system 300 may perform the aforementioned techniques with reference to passengers (e.g., other users 102) of the vehicle 305.

Additionally, or alternatively, the system 300 may adjust one or more operational parameters associated with the vehicle 305 based on physiological data, a physiological state of the user 102, a psychological state of the user 102, or any combination thereof, prior to the user 102 operating the vehicle 305. For example, the system 300 may identify that the user 102 experienced a high stress level and a high activity level throughout a duration of a work day and may decrease a brightness of lights and displays and decrease a volume of music of the vehicle 305. In another example, the system 300 may identify that the user 102 just completed a workout (e.g., based on physiological data) and may decrease a temperature of the vehicle 305. In some cases, as described previously, the system 300 may turn on the vehicle and decrease the temperature of the vehicle 305 prior to the user 102 entering the vehicle 305 (e.g., based on determining the user 102 typically operates the vehicle 10 minutes after completing a workout).

Additionally, or alternatively, the system 300 may adjust one or more operational parameters associated with the vehicle 305 based on information obtained from one or more external sources. For example, the system 300 may communicate with a smart thermostat in a home of the user 102 and may adjust a temperature of the vehicle 305 based on a temperature indicated by the smart thermostat.

Additionally, or alternatively, the system 300 may identify one or more commands associated with controlling the vehicle 305 based on physiological data associated with the user 102. In some cases, the system 300 may identify a gesture performed by a hand of the user 102 wearing the wearable device 104, where the gesture corresponds to a command associated with controlling the vehicle 305. For example, passing a hand of the user 102 wearing the wearable device 104 over a light within the vehicle 305 may turn the light on or off. In another example, the user 102 may perform one or more gestures via a hand of the user 102 associated with controlling a radio of the vehicle 102, such as a first gesture associated with changing a channel, a second gesture associated with skipping to a next song, a third gesture associated with increasing a volume of the radio, a fourth gesture associated with decreasing the volume of the radio, or any combination thereof. In some examples, the command may be based on a speed of the gesture being performed, a location of the gesture being performed, a direction in which the gesture is performed, or any combination thereof. Additionally, or alternatively, the user 102 may interact with the wearable device 104 (e.g., tapping the wearable device 104), where the interaction corresponds to a command associated with controlling the vehicle 305. For example, tapping a finger of the user 102 wearing the wearable device 104 two times on a steering wheel of the vehicle 305 may end a call being broadcast through the vehicle 305.

In some aspects, the vehicle 305 may provide feedback (e.g., haptic feedback, audio feedback, visual feedback, or any combination thereof) to the user 102 via the wearable device 104, the user device 106, or both. For example, the system 300 may receive telemetry data, or data associated with the vehicle 305, from one or more sensors of the vehicle 305 based on the user driving the vehicle 305 (e.g., throughout a time interval). For the purposes of the present disclosure, the term telemetry data may refer to any data collected by sensors of the vehicle 305, such as proximity sensors, GPS sensors, light-based sensors (e.g., light sensors for adjusting headlights), and the like. Additionally, or alternatively, the data collected by sensors of the vehicle 305 may include physiological data collected from the user 102 via the sensors of the vehicle 305. For example, the sensors of the vehicle 305 may include one or more sensors (e.g., temperature sensors, PPG sensors, etc.) on the steering wheel of the vehicle 305, one or more cameras to monitor the user 102 (e.g., eye movement of the user 102), or the like thereof.

In some cases, the system 300 may identify satisfaction of a trigger condition for providing feedback to the user 102 based on the telemetry data associated with the vehicle 305 and, as such, may cause the wearable device 104 to provide feedback to the user 102 based on the satisfying the trigger condition. Additionally, the system 300 may cause the wearable device 104 to provide feedback to the user 102 based on physiological data associated with the user 102. For example, the system 300 may cause the wearable device 104 to provide feedback to the user 102 based on the satisfying the trigger condition and based on identifying that a concentration level (e.g., an engagement status of the user 102 related to driving) is below a threshold.

In some examples, the trigger condition may be associated with a gear shift. That is, the telemetry data may include revolutions per minute (RPM) data associated with an engine of the vehicle 305 and position data associated with a clutch of the vehicle 305. As such, the system 300 may identify that the user 102 should shift gears (e.g., identify an upcoming gear shift event) based on the RPM data associated with the engine and may identify that the user 102 has not shifted gears based on the position data associated with the clutch. As such, the system 300 may cause the wearable device 104 to alert the user 102 to change, or shift, gears. For example, a series of vibrational pulses (e.g., haptic feedback) of the wearable device 104 may be associated with suggesting that the user 102 shifts gears.

Additionally, or alternatively, the trigger condition may be associated with blind spot detection. That is, the telemetry data may include proximity data associated with a surrounding environment of the vehicle 305. As such, the system 300 may identify one or more objects within the surrounding environment of the vehicle 305 based on the proximity data, where the one or more objects are within a threshold proximity of the vehicle 305. Additionally, the system 300 may determine that the one or more objects are located in one or more blind spots of the vehicle 305. As such, the system 300 may cause the wearable device 104 to alert the user 102 that an object is located in at least one blind spot of the vehicle 305. For example, two beeps (e.g., audio feedback) of the wearable device 104 may alert the user 102 that an object is in a blind spot located on a left side of the vehicle 305 (e.g., relative to a position of the driver) and three beeps of the wearable device 104 may alert the user 102 that an object is in a blind spot located on a right side of the vehicle 305.

Additionally, or alternatively, the trigger condition may be associated with navigational directions. That is, the system 300 may receive navigational data associated with a route of the user 102 within the vehicle 305 and may identify an upcoming turn, curve, stop, or the like thereof (e.g., navigational event) based on the telemetry data associated with the vehicle 305 and the navigational data. As such, the system 300 may cause the wearable device 104 to alert the user 102 of the upcoming navigational event. For example, a series of vibrations at an increasing frequency of the wearable device 104 may alert the user 102 that a turn is approaching. In some cases, the wearable device 104 may utilize different haptic feedback patterns to indicate left turns vs. right turns, to indicate upcoming stop, etc.

Additionally, or alternatively, the trigger condition may be associated with crash detection. That is, the system 300 may detect that the vehicle 305 is at risk of collision and may cause the wearable device 104 to alert the user 102 of the risk of collision so that the user 102 may avoid the collision. Additionally, or alternatively, the trigger condition may be associated with a speed of the vehicle 305. For example, the system 300 may detect that the vehicle 305 is moving at a speed exceeding a speed limit (e.g., or at a speed an unsafe amount under the speed limit) and may cause the wearable device 104 to alert the user 102 of the speed of the vehicle so that the user 102 may slow down the vehicle 305 (e.g., or speed the vehicle 305 up). Additionally, or alternatively, the trigger condition may be associated with parking of the vehicle 305. For example, the system 300 may detect that the user 102 is attempting to park the vehicle 305 and may alert the user 102 of the vehicle 305 coming within a threshold proximity to obstacles. In another example, the system 300 may detect that the user 102 is attempting to determine where the vehicle 305 is parked and may provide vibrational alerts to the user 102 indicating that the user 102 is moving towards (e.g., or away from) the vehicle 305 so that the user 102 may locate the vehicle 305.

Additionally, or alternatively, the system 300 may display an indication of physiological data associated with the user 102, a physiological state of the user 102, a psychological state of the user 102, or any combination thereof, via the vehicle 305. For example, the system 300 may display, via the vehicle 305, an indication of a stress level of the user 102, a concentration level of the user 102, or the like thereof. In some cases, the system 300 may display the indication continuously while, in some other cases, the system 300 may display the indication based on a change in the physiological data associated with the user 102, the physiological state of the user 102, the psychological state of the user 102, or any combination thereof.

Additionally, or alternatively, the system 300 may monitor driving of the user 102, habits associated with the user 102 while driving, or the like thereof, based on data collected via the ring 104, telemetry data associated with the vehicle 305, or both. For example, the system 300 may detect that the user 102 is driving based on vibration of the ring 104 while the user 102 is gripping the steering wheel. In another example, the system 300 may detect that the user 102 is taking a break (e.g., relaxing) or that the user 102 did not sleep enough the previous night. In some cases, the system 300 may relay the monitored data (e.g., driving of the user 102, habits associated with the user 102) to a third-party. For example, the user 102 may be a truck driver, such that the monitored data is relayed back to a company that the user 102 works for, such that the company may monitor performance of the user 102. In such cases, the system 300 may further disable or modify one or more operational features of the vehicle 305 based on one or more company-defined policies. For example, the system 300 may disable an ability for the user 102 to drive the vehicle 305 for a predefined duration based on a tiredness level of the user 102 exceeding a threshold (e.g., forcing the user to take a break). In some cases, the system 300 may relay (e.g., transmit) the monitored data via the user device 106, via the vehicle 305 (e.g., the ring 104 is connected to the vehicle 305 via Bluetooth, or the like thereof), or both.

Additionally, or alternatively, the system 300 may identify one or more disruptions to the route of the user 102, such as a vehicular crash, a presence of law enforcement, traffic, or the like thereof, and may alert the user 102 to the one or more disruptions via the wearable device 104. Such disruptions may be determined based on data retrieved from third-party applications (e.g., Google Maps), based on communications exchanged between vehicles 305, and the like. For example, one or more LEDs in the wearable device 104 may flash red, white, and blue (e.g., visual feedback) based on an upcoming presence of police radar on the route of the user 102. In some examples, the system 300 may additionally prompt the user 102 to alter or modify the route based on the one or more disruptions.

Additionally, or alternatively, the system 300 may identify that the vehicle 305 (e.g., associated with the user 102) is in a vehicular crash and may transmit an alert, such as to an emergency contact of the user 102, to emergency services, or the like thereof. In such cases, the alert may include an indication of a location of the user 102 (e.g., and the vehicle 305), information (e.g., details) associated with the crash (e.g., crash magnitude, speed prior to impact, etc.), physiological data associated with the user 102 (e.g., electrocardiogram (ECG), heart rate, blood pressure), information associated with the user 102 (e.g., name, age, sex, weight, etc.), or any combination thereof. Additionally, or alternatively, the system 300 may request a state of the user 102 following the vehicular crash. For example, the system 300 may display a message to the user 102 via the user device 106 stating “We've detected a crash. Do you need medical attention?” As such, the user 102 may respond to the message confirming, or denying, that the user 102 is okay. In some examples, failure to respond to the message may trigger the alert to the emergency contact, the emergency services, or both. In some cases, the system 300 may determine the user 102 is in a vehicular crash based on data associated with the vehicle 305, data associated with the user 102 (e.g., accelerometer data associated with the ring 104), or both. Additionally, or alternatively, the system 300 may adjust one or more operational parameters of the vehicle 305 based on detection of the vehicular crash. For example, the system 300 may unlock doors, decrease a music volume, decrease a temperature of the vehicle 305, or any combination thereof, based on detection of the vehicular crash.

Additionally, or alternatively, the system 300 may determine one or more effects (e.g., correlations) that driving the vehicle 305 has on the user 102 based on the telemetry data associated with the vehicle 305 and the physiological data collected via the wearable device 104. For example, the system 300 may identify one or more driving events or conditions, such as traffic, construction, road conditions, or the like thereof, and identify physiological data collected from the user 102 prior to, during, and following the one or more driving events. As such, the system 300 may identify one or more correlations between the one or more driving events and the physiological data associated with the user 102. For example, the system 300 may identify that stop and go traffic leads to an increase in a heart rate of the user 102 (e.g., due to stress) and causes the user 102 to fatigue (e.g., at a faster rate than without traffic). In another example, the system 300 may determine that the user 102 experiences elevated stress levels while driving in rush-hour traffic relative to driving to leisure activities (e.g., hobbies). In some cases, the system 300 may display, via a GUI of the user device 106, an indication of information associated with the one or more correlations. For example, continuing with the previous example, the system 300 may display an indication to the user 102 stating “Traffic is adding to your stress! Try avoiding traffic on your commute home.” Additionally, or alternatively, the system 300 may display one or more suggestions or recommendations for the user 102 based on the one or more correlations. For example, the system 300 may determine that traffic is increasing a stress level of the user and may suggest for the user 102 to take a break, select an alternate route, perform one or more stress-relieving techniques (e.g., a relaxation session), or any combination thereof.

In some cases, the system 300 may generate one or more maps (e.g., heat maps), a report, or both, based on the one or more correlations between the one or more driving events and the physiological data associated with the user 102. For example, the system 300 may determine that a specific area of a route taken by the user 102 to and from work is associated with an increase in stress level of the user. In another example, the user 102 may be in a high performance racing scenario, such that the system may a report indicating changes in physiological data (e.g., indicative of stress, concentration, etc.) correlated to gear shifts, turns, g-force loads, or the like thereof.

In some examples, the system 300 may identify a driving style of the user 102 based on the one or more correlations. For example, the system 300 may identify one or more recommended (e.g., suited, optimal) driving styles for the user 102 and may display one or more messages (e.g., insights) to the user 102 to guide the user 102 towards the one or more recommended driving styles. Additionally, or alternatively, the system 300 may identify a physiological state of the user 102, a psychological state of the user 102, or both, based on physiological data associated with the user 102 and the driving style of the user 102. For example, the system 300 may identify that that user 102 is breaking hard and taking sharp turns and that a heart rate of the user 102 is elevated resulting in the system 300 determining that the user 102 is stressed.

Additionally, or alternatively, the system 300 may modify one or more future recommended routes for the user 102 based on the one or more correlations. For example, the vehicle 305 may be a self-driving vehicle 305. As such, the vehicle 305 may alter one or characteristics associated with driving the self-driving vehicle 305, such as route, driving habits, driving conditions, or the like thereof, based on the one or more correlations. In another example, the system 300 may determine that the user 102 experiences elevated stress levels while driving in certain geographical locations or on certain road conditions. As such, the system 300 may suggest routes that avoid the geographical locations or road conditions associated with stress.

In some examples, the system 300 may generate a database of correlations associated with multiple users 102 and may modify one or more future recommended routes for the user 102 based on the datable of correlations. For example, the system 300 may identify that a given area along an intended route of the user 102 is associated with high stress (e.g., an average stress level of the multiple users 102 is high in the given area) and may recommend a route avoiding the given area (e.g., even if the system 300 has not identified a high stress level for the user 102 in the given area).

Additionally, or alternatively, the system 300 may support sharing of user-related information between vehicles 305. For example, the system 300 may identify that the user 102 is experiencing a medical event and may transmit an alert to a nearby vehicle 305 alerting the other user 102 of the nearby vehicle 305 of the medical event. In another example, the system 102 may identify that the user 102 is distracted (e.g., is not concentrating, based on HRV of the user 102) and may transmit an alert to nearby vehicles 305 warning the other users 102 of the nearby vehicles 305 that the user 102 is distracted.

In some cases, data associated with the user 102 (e.g., physiological data, user-identifiable data, a physiological state, a psychological state, or the like thereof) transmitted between devices of the system 300 (e.g., the vehicle 305, the ring 104, the user device 106, external devices) may be encrypted to support user privacy. Additionally, or alternatively, the user 102 may be capable of allowing or not allowing transmission of any subset of the data associated with the user 102. For example, the user 102 may allow the system 300 to communicate physiological data associated with the user 102 between the ring 104 and a vehicle 305 operated (e.g., owned) by the user 102 but not with other vehicles 305. Additionally, or alternatively, the system 300 may enable the user 102 to enable or disable any of the techniques described herein.

In some examples, the system 300 may determine whether or how the user 102 is holding a steering wheel of the vehicle 305 based on the physiological data. For example, the ring 104 may include multiple temperature sensors located at different positions around a circumference of the ring 104 (e.g., on a PCB). For example, four temperature sensors may be equally spaced (e.g., at 90 degree increments) around the circumference of the ring 104. In such cases, a first temperature sensor may positioned relative to a top of a finger of the user 102 (e.g., relative to a back of the hand of the user 102, at a “top” position relative to the ring 104), a second temperature sensor may be positioned relative to a first side of the finger of the user 102, a third temperature sensor may be positioned relative to a bottom of the finger of the user 102 (e.g., relative to a palm of the hand of the user 102, at a “bottom” position relative to the ring 104), and a fourth temperature sensor may be positioned relative to a second side of the finger of the user 102.

Thus, in some cases, one or more temperature sensors of the multiple temperature sensors may measure different temperature values. For example, when the ring 104 is worn on a thumb of the user 102 (e.g., without rotation), no other fingers may be adjacent to (e.g., within a threshold distance next to) the thumb, such that the multiple temperature sensors may measure a same temperature value (e.g., within a threshold deviance) due to heat measured by the ring 104 being from the thumb (e.g., only being from the thumb). Conversely, when the ring 104 is worn on a middle finger or a ring finger of the user 102 (e.g., without rotation), the second temperature sensor and the fourth temperature sensor (e.g., positioned relative to the sides of the finger of the user 102) may measure a different temperature value than the first temperature and the third temperature sensor due to multiple fingers adjacent to the middle finger or the ring finger heating the ring 104. In other words, the second temperature sensor and the fourth temperature sensor may measure heat from the middle finger or the ring finger and heat from the adjacent fingers. In another example, when the ring 104 is worn on an index finger or a pink finger of the user 102 (e.g., without rotation), either the second temperature sensor or the fourth temperature sensor may measure a different temperature value than the first temperature, the third temperature sensor, and either the fourth temperature sensor or the second temperature sensor, respectively, due to a single finger adjacent to the index finger or the pink finger heating the ring 104.

Additionally, one or more temperature sensors of the multiple temperature sensors may measure different temperature values based on the user 102 holding an object, such as the steering wheel, that reflects body heat back to the finger of the user 102 such that the one or more temperature sensors measures heat from the finger as well as the heat reflect back off of the object. For example, when the user 102 is holding the steering wheel, the third temperature sensor (e.g., relative to the bottom of the finger of the user 102) may measure a different temperature value than any combination of the first temperature, the second temperature value, and the third temperature sensor due to the user 102 holding the steering wheel. That is, the fourth temperature sensor may measure heat from the finger as well as heat reflected back off from the steering wheel. In some cases, determination of differences in temperature values between multiple temperature sensors, the cause of the differences (e.g., adjacent fingers, holding an object), or both, may be based on one or more of a rotation of the ring 102 relative to the finger of the user 102, a quantity of the multiple temperature sensors, and a location of each temperature sensor of the multiple temperature sensors relative to the ring 104, a finger of the user 102, one or more other temperature sensors of the multiple temperature sensors, or any combination thereof.

In some cases, a concentration level of the user 102 (e.g., as described herein) may further be based on whether or how the user 102 is holding the steering wheel of the vehicle 305. For example, the user 102 not holding the steering wheel may be indicative of a lower concentration level of the user 102 relative to the user 102 holding the steering wheel (e.g., with one hand). In another example, the user 102 holding the steering wheel with one hand may be indicative of a lower concentration level of the user 102 relative to the user 102 holding the steering wheel with two hands (e.g., as detected based on the user 102 wearing a ring 104 on a finger on each hand). Additionally, or alternatively, the system 300 may alert the user 102 when the user 102 is not holding onto the steering wheel (e.g., the user 102 removing one or more of their hands from the steering wheel may be a trigger condition).

Though described in the context of four temperature sensors equally spaced around the circumference of the ring 104, this is not to be regarded as a limitation of the present disclosure. In this regard, any quantity of temperature sensors may be considered with regards to the techniques described herein. Further, the multiple temperature sensors may be located at any position relative to the ring 104 (e.g., symmetrically or asymmetrically)

As described herein, the vehicle 305 may refer to any vehicle 305 capable of transporting the user 102, including, but not limited to, a car, a self-driving car, a motorcycle, a bus, an aircraft (e.g., a plane, a spaceship), a bicycle, a railed vehicle (e.g., a train), or the like thereof.

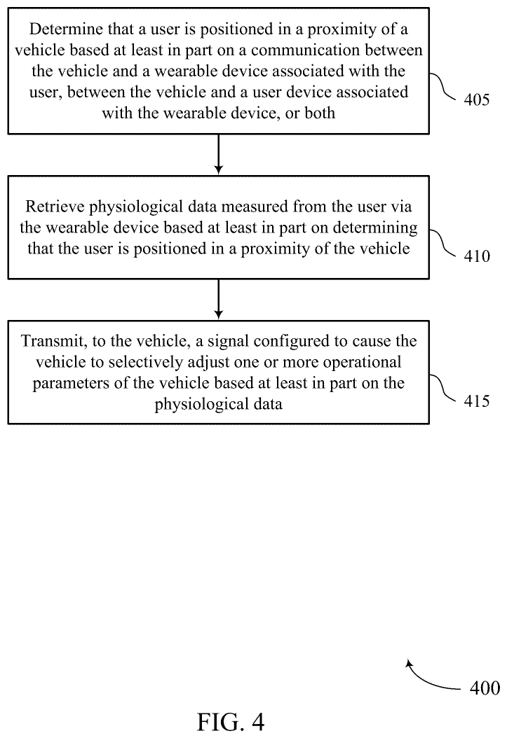

FIG. 4 shows a flowchart illustrating a method 400 that supports techniques for leveraging automotive and wearable-based data in accordance with aspects of the present disclosure. The operations of the method 400 may be implemented by or its components as described herein. For example, the operations of the method 400 may be performed by. In some examples, may execute a set of instructions to control the functional elements of to perform the described functions. Additionally, or alternatively, may perform aspects of the described functions using special-purpose hardware.

At 405, the method may include determining that a user is positioned in a proximity of a vehicle based at least in part on a communication between the vehicle and a wearable device associated with the user, between the vehicle and a user device associated with the wearable device, or both. The operations of block 405 may be performed in accordance with examples as disclosed herein.

At 410, the method may include retrieving physiological data measured from the user via the wearable device based at least in part on determining that the user is positioned in a proximity of the vehicle. The operations of block 410 may be performed in accordance with examples as disclosed herein.

At 415, the method may include transmitting, to the vehicle, a signal configured to cause the vehicle to selectively adjust one or more operational parameters of the vehicle based at least in part on the physiological data. The operations of block 415 may be performed in accordance with examples as disclosed herein.

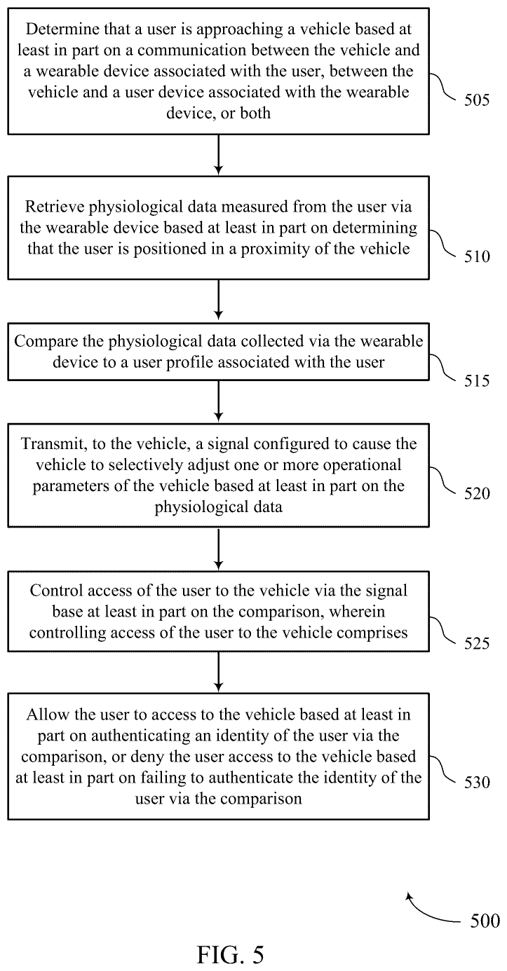

FIG. 5 shows a flowchart illustrating a method 500 that supports techniques for leveraging automotive and wearable-based data in accordance with aspects of the present disclosure. The operations of the method 500 may be implemented by or its components as described herein. For example, the operations of the method 500 may be performed by. In some examples, may execute a set of instructions to control the functional elements of to perform the described functions. Additionally, or alternatively, may perform aspects of the described functions using special-purpose hardware.

At 505, the method may include determining that a user is approaching a vehicle based at least in part on a communication between the vehicle and a wearable device associated with the user, between the vehicle and a user device associated with the wearable device, or both. The operations of block 505 may be performed in accordance with examples as disclosed herein.

At 510, the method may include retrieving physiological data measured from the user via the wearable device based at least in part on determining that the user is positioned in a proximity of the vehicle. The operations of block 510 may be performed in accordance with examples as disclosed herein.

At 515, the method may include comparing the physiological data collected via the wearable device to a user profile associated with the user. The operations of block 515 may be performed in accordance with examples as disclosed herein.

At 520, the method may include transmitting, to the vehicle, a signal configured to cause the vehicle to selectively adjust one or more operational parameters of the vehicle based at least in part on the physiological data. The operations of block 520 may be performed in accordance with examples as disclosed herein.

At 525, the method may include controlling access of the user to the vehicle via the signal based at least in part on the comparison, wherein controlling access of the user to the vehicle comprises. The operations of block 525 may be performed in accordance with examples as disclosed herein.

At 530, the method may include allowing the user to access to the vehicle based at least in part on authenticating an identity of the user via the comparison, or denying the user access to the vehicle based at least in part on failing to authenticate the identity of the user via the comparison. The operations of block 535 may be performed in accordance with examples as disclosed herein.

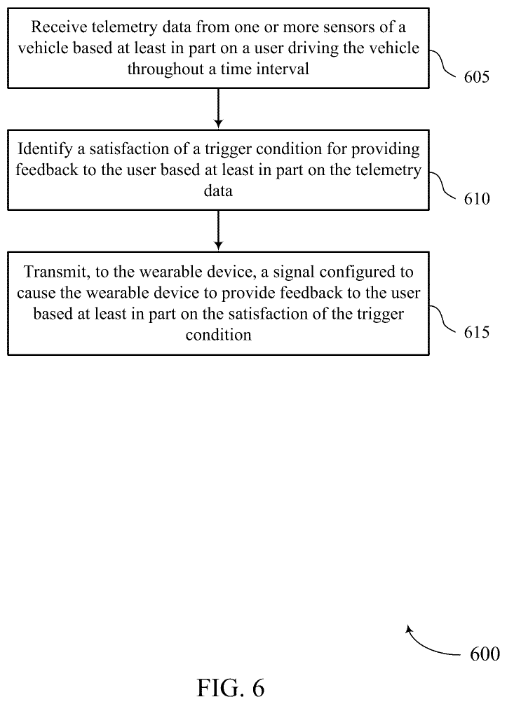

FIG. 6 shows a flowchart illustrating a method 600 that supports techniques for leveraging automotive and wearable-based data in accordance with aspects of the present disclosure. The operations of the method 600 may be implemented by or its components as described herein. For example, the operations of the method 600 may be performed by. In some examples, may execute a set of instructions to control the functional elements of to perform the described functions. Additionally, or alternatively, may perform aspects of the described functions using special-purpose hardware.

At 605, the method may include receiving telemetry data from one or more sensors of a vehicle based at least in part on a user driving the vehicle throughout a time interval. The operations of block 605 may be performed in accordance with examples as disclosed herein.

At 610, the method may include identifying a satisfaction of a trigger condition for providing feedback to the user based at least in part on the telemetry data. The operations of block 610 may be performed in accordance with examples as disclosed herein.

At 615, the method may include transmitting, to the wearable device, a signal configured to cause the wearable device to provide feedback to the user based at least in part on the satisfaction of the trigger condition. The operations of block 615 may be performed in accordance with examples as disclosed herein.

It should be noted that the methods described above describe possible implementations, and that the operations and the steps may be rearranged or otherwise modified and that other implementations are possible. Furthermore, aspects from two or more of the methods may be combined.

A method by an automobile system is described. The automobile system may include a vehicle, a wearable device configured to acquire physiological data from a user, and one or more processors communicatively coupled with the vehicle and the wearable device. In such cases, the method may include determining that the user is positioned in a proximity of the vehicle based at least in part on a communication between the vehicle and the wearable device, between the vehicle and a user device associated with the wearable device, or both, retrieving physiological data measured from the user via the wearable device based at least in part on determining that the user is positioned in a proximity of the vehicle, and transmitting, to the vehicle, a signal configured to cause the vehicle to selectively adjust one or more operational parameters of the vehicle based at least in part on the physiological data.

An automobile system is described. The automobile system may include a vehicle and a wearable device configured to acquire physiological data from a user. Additionally, the automobile system may include one or more memories storing processor executable code, and one or more processors coupled with the one or more memories and processors communicatively coupled with the vehicle and the wearable device. The one or more processors may individually or collectively operable to execute the code to cause the automobile system to determine that the user is positioned in a proximity of the vehicle based at least in part on a communication between the vehicle and the wearable device, between the vehicle and a user device associated with the wearable device, or both, retrieve physiological data measured from the user via the wearable device based at least in part on determining that the user is positioned in a proximity of the vehicle, and transmit, to the vehicle, a signal configured to cause the vehicle to selectively adjust one or more operational parameters of the vehicle based at least in part on the physiological data.

Another automobile system is described. The automobile system may include a vehicle, a wearable device configured to acquire physiological data from a user, and one or more processors communicatively coupled with the vehicle and the wearable device. Additionally, the automobile system may include means for determining that the user is positioned in a proximity of the vehicle based at least in part on a communication between the vehicle and the wearable device, between the vehicle and a user device associated with the wearable device, or both, means for retrieving physiological data measured from the user via the wearable device based at least in part on determining that the user is positioned in a proximity of the vehicle, and means for transmitting, to the vehicle, a signal configured to cause the vehicle to selectively adjust one or more operational parameters of the vehicle based at least in part on the physiological data.

A non-transitory computer-readable medium storing code associated with an automobile system is described, where the automobile system includes a vehicle, a wearable device configured to acquire physiological data from a user, and one or more processors communicatively coupled with the vehicle and the wearable device. The code may include instructions executable by a processor to determine that the user is positioned in a proximity of the vehicle based at least in part on a communication between the vehicle and the wearable device, between the vehicle and a user device associated with the wearable device, or both, retrieve physiological data measured from the user via the wearable device based at least in part on determining that the user is positioned in a proximity of the vehicle, and transmit, to the vehicle, a signal configured to cause the vehicle to selectively adjust one or more operational parameters of the vehicle based at least in part on the physiological data.

Some examples of the method, apparatus, and non-transitory computer-readable medium described herein may further include operations, features, means, or instructions for determining that the user may be approaching the vehicle, comparing the physiological data collected via the wearable device to a user profile associated with the user, controlling access of the user to the vehicle via the signal based at least in part on the comparison, wherein controlling access of the user to the vehicle comprises, allowing the user to access to the vehicle based at least in part on authenticating an identity of the user via the comparison, and denying the user access to the vehicle based at least in part on failing to authenticate the identity of the user via the comparison.

Some examples of the method, apparatus, and non-transitory computer-readable medium described herein may further include operations, features, means, or instructions for receiving an indication of a destination of the user and determining a navigational route from a current position of the user to the destination based at least in part on the physiological data.

Some examples of the method, apparatus, and non-transitory computer-readable medium described herein may further include operations, features, means, or instructions for receiving an indication of an incoming message, an incoming call, or both and determining whether or not to cause one or more components of the vehicle to alert the user of the incoming message, the incoming call, or both, based at least in part on the physiological data and based at least in part on determining that the user may be driving the vehicle.

Some examples of the method, apparatus, and non-transitory computer-readable medium described herein may further include operations, features, means, or instructions for determining that the user may be experiencing an altered state of consciousness based at least in part on the physiological data, wherein the signal may be configured to cause the vehicle to prevent the user from operating the vehicle.

In some examples of the method, apparatus, and non-transitory computer-readable medium described herein, the one or more operational parameters of the vehicle comprise a vehicle seat setting, a mirror setting, a vehicle climate control setting, an interior lighting setting, a driving performance profile associated with the vehicle, or any combination thereof.

A method by an automobile system is described. The automobile system may include a vehicle, a wearable device configured to acquire physiological data from a user, and one or more processors communicatively coupled with the vehicle and the wearable device. In such cases, the method may include receiving telemetry data from one or more sensors of the vehicle based at least in part on the user driving the vehicle throughout a time interval, identifying a satisfaction of a trigger condition for providing feedback to the user based at least in part on the telemetry data, and transmitting, to the wearable device, a signal configured to cause the wearable device to provide feedback to the user based at least in part on the satisfaction of the trigger condition.

An automobile system is described. The automobile system may include a vehicle and a wearable device configured to acquire physiological data from a user. Additionally, the automobile system may include one or more memories storing processor executable code, and one or more processors coupled with the one or more memories and processors communicatively coupled with the vehicle and the wearable device. The one or more processors may individually or collectively operable to execute the code to cause the automobile system to receive telemetry data from one or more sensors of the vehicle based at least in part on the user driving the vehicle throughout a time interval, identify a satisfaction of a trigger condition for providing feedback to the user based at least in part on the telemetry data, and transmit, to the wearable device, a signal configured to cause the wearable device to provide feedback to the user based at least in part on the satisfaction of the trigger condition.

Another automobile system is described. The automobile system may include a vehicle, a wearable device configured to acquire physiological data from a user, and one or more processors communicatively coupled with the vehicle and the wearable device. Additionally, the automobile system may include means for receiving telemetry data from one or more sensors of the vehicle based at least in part on the user driving the vehicle throughout a time interval, means for identifying a satisfaction of a trigger condition for providing feedback to the user based at least in part on the telemetry data, and means for transmitting, to the wearable device, a signal configured to cause the wearable device to provide feedback to the user based at least in part on the satisfaction of the trigger condition.

A non-transitory computer-readable medium storing code associated with an automobile system is described, where the automobile system includes a vehicle, a wearable device configured to acquire physiological data from a user, and one or more processors communicatively coupled with the vehicle and the wearable device. The code may include instructions executable by a processor to receive telemetry data from one or more sensors of the vehicle based at least in part on the user driving the vehicle throughout a time interval, identify a satisfaction of a trigger condition for providing feedback to the user based at least in part on the telemetry data, and transmit, to the wearable device, a signal configured to cause the wearable device to provide feedback to the user based at least in part on the satisfaction of the trigger condition.

In some examples of the method, apparatus, and non-transitory computer-readable medium described herein, and the method, apparatuses, and non-transitory computer-readable medium may include further operations, features, means, or instructions for identifying an upcoming gear shift event based at least in part on the RPM data and the clutch position data, wherein the satisfaction of the trigger condition may be based at least in part on the upcoming gear shift event.

In some examples of the method, apparatus, and non-transitory computer-readable medium described herein, and the method, apparatuses, and non-transitory computer-readable medium may include further operations, features, means, or instructions for identifying one or more objects within the surrounding environment of the vehicle based at least in part on the proximity data and determining that the one or more objects may be positioned within one or more blind spots of the vehicle, wherein the satisfaction of the trigger condition may be based at least in part on the one or more objects being positioned within the one or more blind spots.

Some examples of the method, apparatus, and non-transitory computer-readable medium described herein may further include operations, features, means, or instructions for receiving navigational data associated with a navigational route of the user within the vehicle and identifying an upcoming navigational event based at least in part on the telemetry data and the navigational route, wherein the satisfaction of the trigger condition may be based at least in part on the upcoming navigational event.

Some examples of the method, apparatus, and non-transitory computer-readable medium described herein may further include operations, features, means, or instructions for receiving navigational data associated with a navigational route of the user within the vehicle and identifying one or more interruptions along the navigational route, wherein the one or more interruptions comprise a vehicular crash, traffic, a presence of law enforcement, or any combination thereof, wherein the satisfaction of the trigger condition may be based at least in part on the one or more interruptions.

Some examples of the method, apparatus, and non-transitory computer-readable medium described herein may further include operations, features, means, or instructions for determining one or more effects that driving the vehicle may have on the user based at least in part on the telemetry data and the physiological data and causing a graphical user interface of a user device associated with the user to display information associated with the one or more effects.

In some examples of the method, apparatus, and non-transitory computer-readable medium described herein, the feedback provided by the wearable device comprises haptic feedback, audio feedback, visual feedback, or any combination thereof.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herei

What is claimed is:

1. A wearable system communicatively coupled with a vehicle, comprising:
a finger-worn wearable ring device configured to acquire physiological data from a user; and
one or more processors communicatively coupled with the vehicle and the finger-worn wearable ring device, the one or more processors configured to:
receive telemetry data from one or more sensors of the vehicle based, at least in part, on the user driving the vehicle during a time interval;
identify a satisfaction of at least one trigger condition for providing feedback to the user based, at least in part, on the telemetry data, current physiological data acquired from the user, or both; and
transmitting, to the finger-worn wearable ring device, a signal configured to cause the finger-worn wearable ring device to provide feedback to the user based at least in part on the identified satisfaction of the at least one trigger condition and based, at least in part, on the current physiological data acquired from the user.

2. The wearable system of claim 1, wherein the one or more processors are further configured to:
identify a concentration level of the user based at least in part on the current physiological data, wherein transmitting the signal is based at least in part on the concentration level of the user being below a threshold concentration level.

3. The wearable system of claim 2, wherein, to identify the concentration level of the user, the one or more processors are further configured to:
compare the current physiological data associated with the user to a baseline physiological data associated with the user, wherein the concentration level of the user is based at least in part on a difference between the current physiological data and the baseline physiological data.

4. The wearable system of claim 1, wherein the one or more processors are further configured to:
identify a physiological state, a psychological state, or both, of the user based at least in part on the current physiological data, wherein satisfaction of the at least one trigger condition is based at least in part on the physiological state, the psychological state, or both, of the user being from a set of physiological state, a set of psychological states, or both.

5. The wearable system of claim 4, wherein the feedback provided by the finger-worn wearable ring device comprises an indication of the current physiological data associated with the user, an indication of the physiological state of the user, an indication of the psychological state of the user, one or recommendations to change the physiological state of the user, one or more recommendations to change the psychological state of the user, or any combination thereof.

6. The wearable system of claim 1, wherein the one or more processors are further configured to:
identify that the vehicle was involved in a crash based at least in part on the telemetry data, wherein the satisfaction of the at least one trigger condition is based at least in part on identification of the crash.

7. The wearable system of claim 6, wherein, to transmit the signal configured to cause the finger-worn wearable ring device to provide feedback to the user, the one or more processors are further configured to:
transmit the signal to cause the finger-worn wearable ring device to prompt the user to contact emergency services based at least in part on identification of the crash.

8. The wearable system of claim 7, wherein the one or more processors are further configured to:
transmitting, to the vehicle, a user device associated with the finger-worn wearable ring device, or both, a second signal configured to cause the vehicle, the user device, or both, to transmit an alert to emergency services, one or more emergency contacts associated with the user, or both, based at least in part on identification of the crash, wherein the alert comprises an indication of the crash, an indication of the current physiological data of the user, an indication of a location of the vehicle, an indication of a current state of the vehicle, or any combination thereof.

9. The wearable system of claim 1, wherein the telemetry data comprises revolution per minute (RPM) data associated with an engine of the vehicle, and clutch position data of the vehicle, wherein the one or more processors are further configured to:
identify an upcoming gear shift event based at least in part on the RPM data and the clutch position data, wherein the satisfaction of the at least one trigger condition is based at least in part on the upcoming gear shift event.

10. The wearable system of claim 1, wherein the telemetry data comprises proximity data associated with a surrounding environment of the vehicle, wherein the one or more processors are further configured to:
identify one or more objects within the surrounding environment of the vehicle based at least in part on the proximity data; and
determine that the one or more objects are positioned within one or more blind spots of the vehicle, wherein the satisfaction of the at least one trigger condition is based at least in part on the one or more objects being positioned within the one or more blind spots.

11. The wearable system of claim 1, wherein the one or more processors are further configured to:
receive navigational data associated with a navigational route of the user within the vehicle; and
identify an upcoming navigational event based at least in part on the telemetry data and the navigational route, wherein the satisfaction of the at least one trigger condition is based at least in part on the upcoming navigational event.

12. The wearable system of claim 1, wherein the one or more processors are further configured to:
receive navigational data associated with a navigational route of the user within the vehicle; and
identify one or more interruptions along the navigational route, wherein the one or more interruptions comprise a vehicular crash, traffic, a presence of law enforcement, or any combination thereof, wherein the satisfaction of the at least one trigger condition is based at least in part on the one or more interruptions.

13. The wearable system of claim 1, wherein the one or more processors are further configured to:
determine one or more effects that driving the vehicle has on the user based at least in part on the telemetry data and the current physiological data; and
causing a graphical user interface of a user device associated with the user to display information associated with the one or more effects.

14. The wearable system of claim 13, wherein the one or more effects that driving the vehicle has on the user comprises a change in physiological state of the user, a change in psychological state of the user, or both, based at least in part on the telemetry data, wherein the change in physiological state of the user, the change in psychological state of the user, or both, are based at least in part on the current physiological data.

15. The wearable system of claim 1, wherein the one or more processors are further configured to:
determine whether the user is holding onto a steering wheel of the vehicle based at least in part on temperature data associated with the user, wherein the current physiological data comprises the temperature data, and wherein satisfaction of the at least one trigger condition is based at least in part on determining the user is not holding onto the steering wheel.

16. The wearable system of claim 15, wherein, to determine whether the user is holding onto the steering wheel, the one or more processors are further configured to:
identify a plurality of temperature values associated with the user, wherein each temperature values of the plurality of temperature values is associated with a different position on the finger-worn wearable ring device relative to a finger of the user; and
identifying one or more differences in temperature between at least a subset of the plurality of temperature values, wherein determining whether the user is holding onto the steering wheel is based at least in part on the one or more differences in temperature.

17. The wearable system of claim 1, wherein the feedback provided by the finger-worn wearable ring device comprises haptic feedback, audio feedback, visual feedback, or any combination thereof.

18. The wearable system of claim 1, wherein the feedback provided by the finger-worn wearable ring device comprises an indication of the current physiological data associated with the user.
